(19) United States
(12) Patent Application Publication
(10) Pub. No.: US 2007/0060992 A1
(43) Pub. Date: Mar. 15, 2007

(54) Methods and devices for mapping the ventricle for pacing lead placement and therapy delivery

(76) Inventor: Carlo Pappone, Milano (IT)

Correspondence Address: Edward Renner, 7700 Bonhomme, Suite 400, St. Louis, MO, 63105, US

(21) Appl. No.: 11/445,921

(22) Filed: Jun. 2, 2006

Related U.S. Application Data
(60) Provisional application No. 60/686,785, filed on Jun. 2, 2005.

(51) Int. Cl. A61N 1/05

(52) U.S. Cl. 607/122

(57) ABSTRACT

A method of placing a pacing lead in the heart includes moving an electrode catheter successively to a plurality of possible placement sites. The viability of the tissue at each site is determined. If the tissue at the site is viable, a pacing signal is applied to the tissue at the site, and the effectiveness of the pacing from the site is measured. After the area has been mapped in this fashion, at least one pacing lead is placed from at least one of the sites which exceeded a predetermined level of pacing effectiveness.

### CROSS-REFERENCE TO RELATED APPLICATIONS

This application claims the benefit of U.S. Provisional Patent Application Ser. No. 60/686,785, filed Jun. 2, 2006, the entire disclosure of which is incorporated herein by reference.

### BACKGROUND OF THE INVENTION

This invention relates to bi-ventricular pacing, and in particular to the placement of pacing leads for bi-ventricular pacing.

Bi-Ventricular pacing has been shown to improve cardiac function in heart failure patients with ventricular de-synchrony by pacing both ventricles using right ventricular and left ventricular pacing leads in such a fashion as to improve hemodynamic function. Typically the leads are individually positioned in the ventricle, and tested to determine whether pacing from that location is acceptable, and if so, the lead is left in place. While this results in a functional placement, it does not result in the optimal placement of the leads.

### SUMMARY OF THE INVENTION

Some embodiments of the method of this invention provide for improved placement of pacing leads in the heart, and in particular in the ventricles. The embodiments employ an advanced device and technique for the interrogation and testing of potential pacing locations to optimize heart function. Generally, a method of placing pacing leads in accordance with this invention comprises moving an electrode catheter successively to a plurality of possible placement sites in the heart. At each site a determination is made whether the tissue at the site is viable. If the tissue at the site is viable, a pacing signal is applied to the tissue at the site, and the effectiveness of pacing from the location is measured. This is repeated over a region of the heart until one or more locations of optimum pacing are determined. The pacing lead can then be placed in the optimum location identified.

Thus, methods in accordance with the preferred embodiments of the present invention facilitate the placement of pacing leads, and in at least some embodiments permit placement of pacing leads at better locations than current methods of lead placement, which merely seek functional locations. These and other features and advantages will be in part apparent and in part pointed out hereinafter.

### BRIEF DESCRIPTION OF THE DRAWINGS

FIG. 1 is a flow chart illustrating the method of mapping the left ventricle to select the location for pacing lead placement in accordance with the principles of this invention;

FIG. 2 is a schematic diagram of a first embodiment of an electrophysiology catheter device useful in various embodiments of the methods of this invention;

FIG. 3 is a schematic diagram of a second embodiment of an electrophysiology catheter device useful in various embodiments of the methods of this invention;

FIG. 4 is a schematic diagram of a third embodiment of an electrophysiology catheter device useful in various embodiments of the methods of this invention;

FIG. 5 is a schematic diagram of a magnetically navigable electrophysiology catheter useful in various embodiments of the methods of the invention;

FIG. 6 is a schematic diagram of a fourth embodiment of an electrophysiology catheter device useful in various embodiments of the methods of this invention;

FIG. 7 is a schematic diagram illustrating various electrode configurations applicable to the catheters shown in FIGS. 2-6;

FIG. 8 is a schematic diagram showing a locator catheter in the left ventricle and a magnetic catheter for sensing and pacing that is placed epicardially in the coronary venous vasculature.

FIG. 9 is an of x-ray images showing a contrast-enhanced images of the vasculature;

FIG. 10 is a schematic diagram showing a contrast-enhanced images of the vasculature;

FIG. 11 is a schematic diagram showing multiple pacing catheters could be navigated and placed in multiple locations; and

FIG. 12 is a schematic diagram showing bipolar ablation; and

FIG. 13 is a schematic diagram showing bipolar ablation

Corresponding reference numerals indicate corresponding parts throughout the several views of the drawings.

### DETAILED DESCRIPTION OF THE PREFERRED EMBODIMENTS

The methods of the preferred embodiments of this invention facilitate the placement of cardiac pacing leads, and in particular the placement of pacing leads for bi-ventricular pacing of the heart. Generally, the method of the preferred embodiments provide for electrically mapping a portion of the heart (preferably the ventricle) via the coronary vasculature using leads or catheters to find optimal pacing locations for chronic pacing lead placement to support resynchronization therapy.

The methods of the preferred embodiments involve the evaluation of the viability of the tissue at various possible pacing locations and the evaluation of pacing at those locations, for example using pressure-volume loops and/or intracardiac electrical activity. The physician directs the lead or catheter to a location in the coronary vasculature and “maps” the area to ensure that the myocardium within proximity of the electrode location is viable. If the tissue at a location is viable, the physician undertakes a pacing protocol at the location and measures the impact of pacing from the location on the physiology of the patient by observing changes in pressure-volume loops and/or intracardiac activity. The physician records the data and then directs the lead or catheter to a new location within the vasculature and repeats the mapping and pacing protocol. By testing several locations in this fashion, the physician can determine the best location or locations for the placement of a chronic pacing lead.

In some embodiments of the methods, the user directs the lead or catheter in an essentially manual operation through the coronary vasculature. In other embodiments of the methods, the user directs the lead or catheter using a robotic system or other remote navigation system. In still other embodiments of the methods, the robotic system is based on mechanical pull wires, rods and/or pulleys. In still other embodiments of the methods, the robotic system is a magnetic system that directs magnetic instruments inside of the body by using externally applied magnetic fields.

The system or the physician can select the single best site for placement of a lead and the physician can implant the lead there. Alternatively, the system or the physician can identify selects several optimal sites, and the physician can place several chronic leads. These leads can then be attached to an implantable device and a pacing sequence can be programmed to optimize the function of the ventricles, pacing each location in parallel or serially in a phased approach to mimic the natural conduction of a healthy ventricle.

The devices used are preferably on the order of about 0.5 French-7 French, with at least one pace/sense electrode adjacent the distal end. In some embodiments, there is a lumen in the center so that a guide wire can be inserted into the tip and this guide wire provides body to the shaft and steerability to the tip. A steering mechanism can be provided, such as manually controlled pull wires or a robotically controlled mechanical or magnetic system that controls the tip of the guide wire. In other embodiments the device can include at least one magnetically responsive element, preferably attached to the tip via a flexible member. The magnetically responsive element, and thus the distal tip of the device, can be oriented by an externally applied magnetic field, so that it can be directed by the user changing the magnetic field.

The pace/sense electrode configurations may include at least two recording electrodes on the tip placed so that the physician may record in a bipolar fashion. Other embodiments of the devices have an electrode placed on the proximal shaft sufficiently far away from the tip electrodes to enable the physician to record unipolar signals.

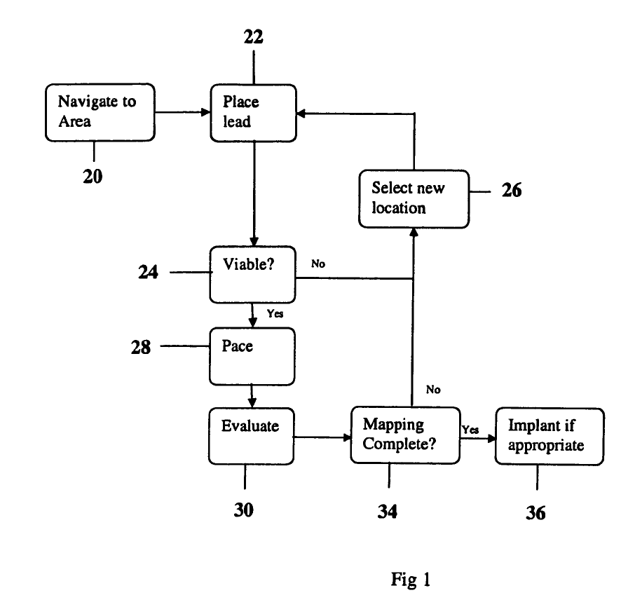

A preferred embodiment of the methods of this invention is shown in FIG. 1. At step 20, the distal end of the catheter is navigated to an area of possible placement. At step 22, the lead (electrode) is placed. At step 24 the area is mapped to determine whether the tissue at the location is viable. If the tissue is not viable, at step 26 a new location is selected, and the process starts over at step 22. If the tissue is viable, then at step 28 pacing is started from the location. At step 30 the pacing is evaluated. After the evaluation of the pacing, at step 32, it is determined whether the mapping is complete, and if not, then at step 26 a new location is selected, and the processes starts over at step 22. If the mapping of the area is complete, then at step 34 one or more implantation locations are selected, and the leads are implanted in the selected locations.

The method of this preferred embodiment can be advantageously conducted with a remote navigation system, and in particular an automated remote navigation system such as an automated magnetic navigation system, available from Stereotaxis, Inc., St. Louis, Mo. Such an automated system can move the leads to each of a plurality of locations in a preplanned pattern, such as a grid or a spiral. Such a system could also be programmed to selected locations intelligently, for example avoiding locations where the tissue can be predicted to be unviable based on locations where the tissue has already been determined to be unviable, or to locations predicted to be effective pacing locations based on locations that have already been determined to be effective pacing locations.

The step of determining the viability of tissue in the location can include sensing local electrical activity or some other method for determining tissue viability.

The step of evaluating the pacing from a particular location can include pressure-volume loops and/or intracardiac electrical activity or some other method for evaluating pacing effectiveness.

After a plurality of locations have been evaluated, the pacing lead can be implanted in a preferred location or preferred locations. The preferred locations are preferably the optimum or near optimum locations. While in the preferred embodiment of this method, the location(s) in the mapped area with the best pacing function are identified, a physician may nonetheless choose (or the system may help the physician choose) to implant the pacing lead at an alternative site that is less than optimum. For example, the location may be selected based on surrounding tissue viability and security of the lead, provided that this still provides some threshold level of pacing activity.

Devices are disclosed herein that can be used to map the vasculature in accordance with the methods of the preferred embodiment. These devices can include a connector on the proximal end with electrodes for connection to a recording system, a proximal shaft and a distal tip with a plurality of pace/sense electrodes located on the tip and shaft for the mapping of the vasculature. Provision is made to steer the devices to enable the device to be directed to a plurality of locations within the vasculature located in the ventricle and base of the heart, typically accessed via the coronary sinus.

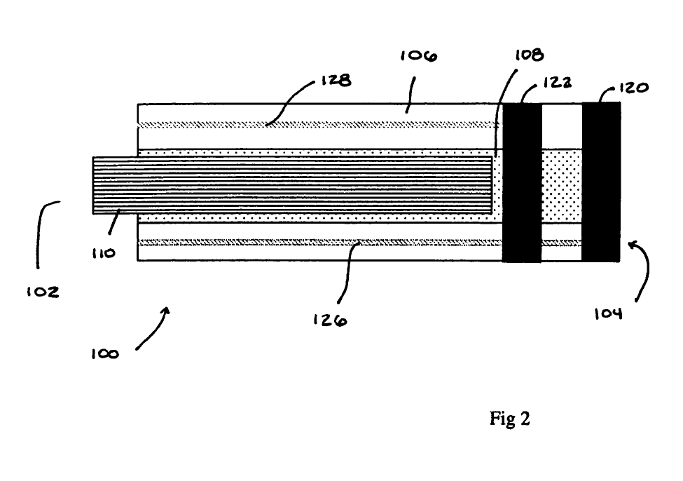

A first embodiment of a device useful in at least some of the preferred embodiments of the methods of this invention is indicated generally as 100 in FIG. 2. The device 100 has a proximal end 102, a distal end 104, and a sidewall 106 forming lumen 108 extending therebetween. In the preferred embodiment the lumen 108 is adapted to receive and pass a guide wire 110 for facilitating the navigation of the device 100. There are preferably two ring electrodes 120 and 122 on the distal end of 104 of the device. The electrodes 120 and 122 may be positioned at the distal end of the device 100. The electrode 122 is positioned proximal to, and spaced from, the electrode 120. Conductors 126 and 128 extend from the electrodes 120 and 122, respectively through the wall 106 of the device 100 to the proximal end where they can be connected to suitable equipment for sensing signals between the electrodes 120 and 122 and for applying a pacing signal between the electrodes 120 and 122.

The guide wire 110 can be navigated to a desired location, such as the right ventricle, and the device 100 advanced over the guide wire. Alternatively the guide wire 110 can be advanced from the distal end of the device 100, and navigated toward the desired location, and then the device 100 can be advanced over the guide wire. The guide wire 110 is again advanced, followed by the device 100, and in this manner the distal end of the device is gradually navigated to the desired location.

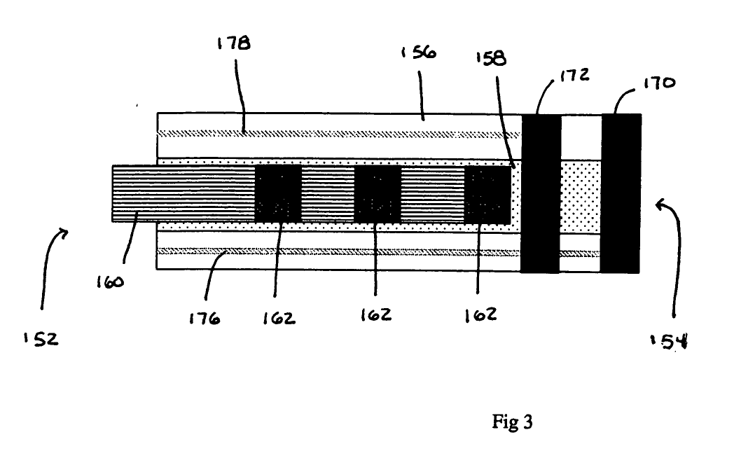

A second embodiment of a device useful in at least some of the preferred embodiments of the methods this invention is indicated generally as 150 in FIG. 3. The device 150 has a proximal end 152, a distal end 154, and a sidewall 156 forming lumen 158 extending therebetween. In the preferred embodiment the lumen 158 is adapted to receive and pass a guide wire 160 for facilitating the navigation of the device 150. The guide wire 160 can have one or more magnetically responsive elements 162 thereon. These elements 162 can be made from a permanent magnetic material or a permeable magnetic material of sufficient size and shape that it tends to align the distal end of the guide wire 160 relative to an externally applied magnetic field. There are preferably two ring electrodes 170 and 172 on the distal end of 154 of the device 150. The electrode 170 may be positioned at the distal end of the device 150. The electrode 172 is positioned proximal to, and spaced from, the electrode 170. Conductors 176 and 178 extend from the electrodes 170 and 172, respectively through the wall 156 of the device 150 to the proximal end 152 where they can be connected to suitable equipment for sensing signals between the electrodes 170 and 172 and for applying a pacing signal between the electrodes 170 and 172.

The guide wire 160 can be navigated to a desired location, such as the right ventricle, and the device 150 advanced over the guide wire. The guide wire 160 can be oriented by applying a magnetic field from an external source magnet, which causes the magnetically responsive elements 162 to align relative to the direction of the applied field. Alternatively the guide wire 160 can be advanced from the distal end 154 of the device 150, and navigated toward the desired location, and then the device 150 can be advanced over the guide wire. The guide wire 160 is again oriented and advanced, followed by the device 150, and in this manner the distal end of the device is gradually navigated to the desired location. In yet another alternative, the guide wire can be left in the lumen 158 of the device 150, so that the magnetically responsive elements 162 are disposed inside the device 150. The application of a magnetic field acts on the magnetic elements 162 on the guide wire 160, orienting the distal end of the device 150.

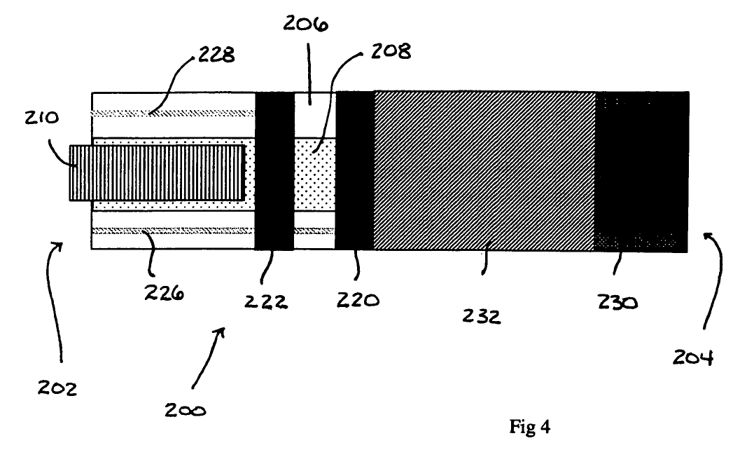

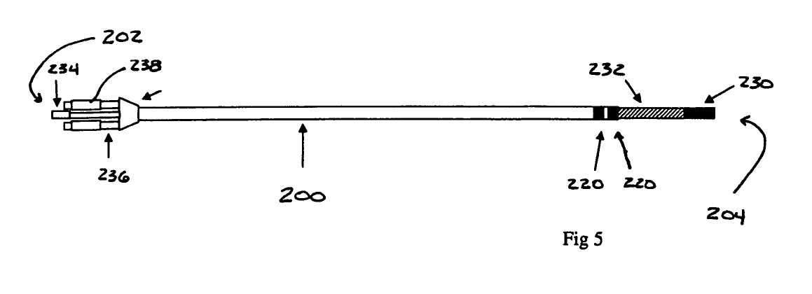

A third embodiment of a device useful in at least some of the preferred embodiments of the methods this invention is indicated generally as 200 in FIGS. 4 and 5. The device 200 has a proximal end 202, a distal end 204, and a sidewall 206 forming lumen 208 extending from the proximal end to a point proximal to the distal end 204. In the preferred embodiment the lumen 208 is adapted to receive a guide wire 210 for facilitating the navigation of the device 200, the guide wire 210 can function to engage and push the distal end of the device 200. In addition, or alternatively, the guide wire 210 may function to stiffen at least the distal portion of the device 200. The guide wire 210 can optionally have one or more magnetically responsive elements (not shown) thereon. These elements can be made from a permanent magnetic material or a permeable magnetic material of sufficient size and shape that it tends to align the distal end of the guide wire 210 relative to an externally applied magnetic field. Thus when the guide wire is disposed in the lumen of the device 200, it enhances the magnetic responsiveness due to the presence of the magnetically responsive elements in the lumen 208.

There are preferably two ring electrodes 220 and 222 adjacent the distal end 204 of the device. The electrode 220 is spaced proximal to the distal end 204, and the electrode 222 is positioned proximal to, and spaced from, the electrode 220. Conductors 226 and 228 extend from the electrodes 220 and 222, respectively, through the wall 206 of the device 200 to the proximal end where they can be connected suitable equipment for sensing signals between the electrodes 220 and 222 and for applying a pacing signal between the electrodes 220 and 222.

There is preferably a magnetically responsive element 230 attached to a flexible element such as a coil 232 forming the distal end 204 of the device 200. The magnetically responsive element 230 can be made from a permanent magnetic material or a permeable magnetic material of sufficient size and shape that it tends to align the distal end of the guide wire relative to an externally applied magnetic field. The coil 232 provides flexibility and a smooth transition between magnetically responsive element 230 and the remainder of the device 200.

The distal end of the device can be oriented by applying a magnetic field from an external source magnet, which causes the magnetically responsive element 230 to move relative to the direction of the applied field. The guide wire 210 can be inserted into the lumen 208 to stiffen the device 200 and to apply a pushing force to the distal end of the device to advance the device in its selected orientation.

As shown in FIG. 5, but applicable to all of the embodiments of the devices described herein, the proximal end 202 of the device 200 can have a sleeve 234 for the introduction of the guide wire 210 into the lumen 208. There are also connectors 236 and 238 for connecting the conductors 226 and 228, to make electrical connections to the ring electrodes 220 and 222.

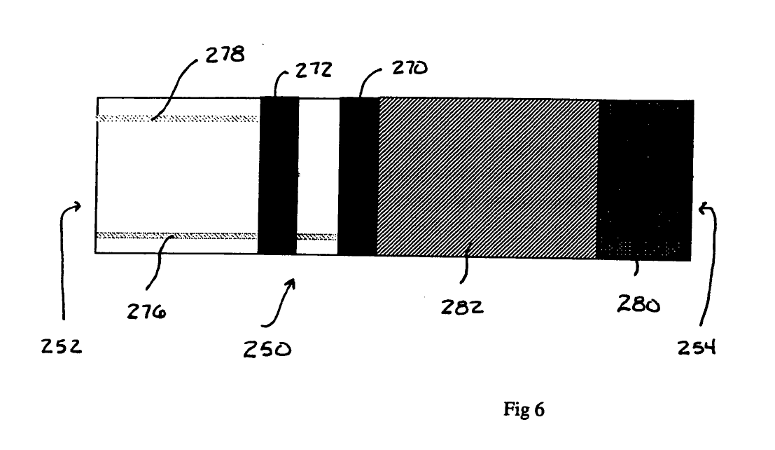

A fourth embodiment of a device useful in at least some of the preferred embodiments of the methods this invention is indicated generally as 250 in FIG. 6. The device 250 has a proximal end 252 and a distal end 254. There are preferably two ring electrodes 270 and 272 adjacent the distal end 254 of the device. The electrode 270 is spaced proximal to the distal end 254, and the electrode 272 is positioned proximal to, and spaced from, the electrode 270. Conductors 276 and 278 extend from the electrodes 270 and 272, respectively, through the device 250 to the proximal end where the can be connected suitable equipment for sensing signals between the electrodes 270 and 272 and for applying a pacing signal between the electrodes 270 and 272.

There is preferably a magnetically responsive element 280 attached to a flexible element such as a coil 282 forming the distal end 254 of the device 250. The magnetically responsive element 280 can be made from a permanent magnetic material or a permeable magnetic material of sufficient size and shape that it tends to align the distal end of the guide wire relative to an externally applied magnetic field. The coil 282 provides flexibility and a smooth transition between magnetically responsive element 280 and the remainder of the device 250.

The distal end of the device can be oriented by applying a magnetic field from an external source magnet, which causes the magnetically responsive elements 280 to move relative to the direction of the applied field.

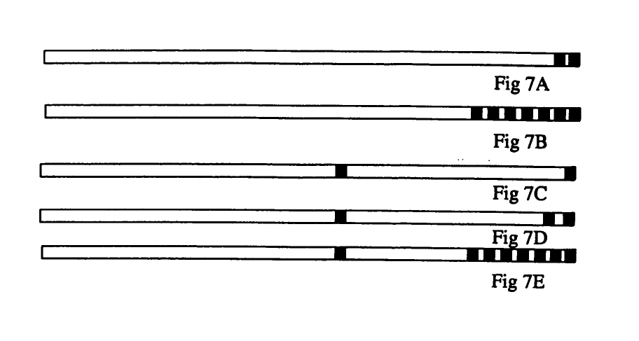

As shown in FIG. 7, the electrodes on the devices 50, 100, 150, 200, and 250 could be arranged in a variety of different configurations. As shown in FIG. 7A, the device could have two electrodes, disposed adjacent the distal end of the device. As shown in FIG. 7B, the device could have multiple electrodes (e.g., 7 electrodes as shown in the Figure), which provide 6 adjacent pairs of electrodes at intervals along the distal end portion of the device. As shown in FIG. 7C, the device could have two electrodes, one disposed adjacent the distal end of the device, and one disposed substantially spaced from the distal end of the device. As shown in FIG. 7D, the device could have three electrodes, two disposed adjacent the distal end of the device, forming a spaced electrode pair, and another spaced substantially from the electrode pair. As shown in FIG. 7E, the device could have multiple electrodes (e.g. 8 electrodes as shown in the Figure), which provides six adjacent pairs of electrodes at intervals along the distal end portion of the device, and another spaced substantially from the six electrodes to operate alternatively as a multipolar electrode or a unipolar electrode.

Operation

In operation, a device, such as one of the devices 50, 100, 150, 200 or 250, is navigated through the vasculature and into the chamber of the heart where the lead will be placed. The electrode is navigated to a first location in the surface of the heart. A determination is made whether the tissue at that location is viable. One way of doing this is to measure electrical activity at the location. If the tissue at the location is viable, then pacing is commenced from the location. During this pacing electrical signals are delivered to the heart from the location, and the results are monitored to gauge the effectiveness of the pacing from this location. Another location is selected, the device is moved to the new location, and the process of determining viability and gauging the effectiveness of pacing from the location is repeated. These steps are repeated until the entire area of interest has been sufficiently mapped.

After the mapping is complete, the data can be processed, or the physician can select one or more locations to return to for lead placement. While the mapping will reveal the location(s) with the maximum pacing effectiveness, these points may not be selected in favor of locations with nearly the same pacing effectiveness but which are better for attaching and maintaining the pacing leads.

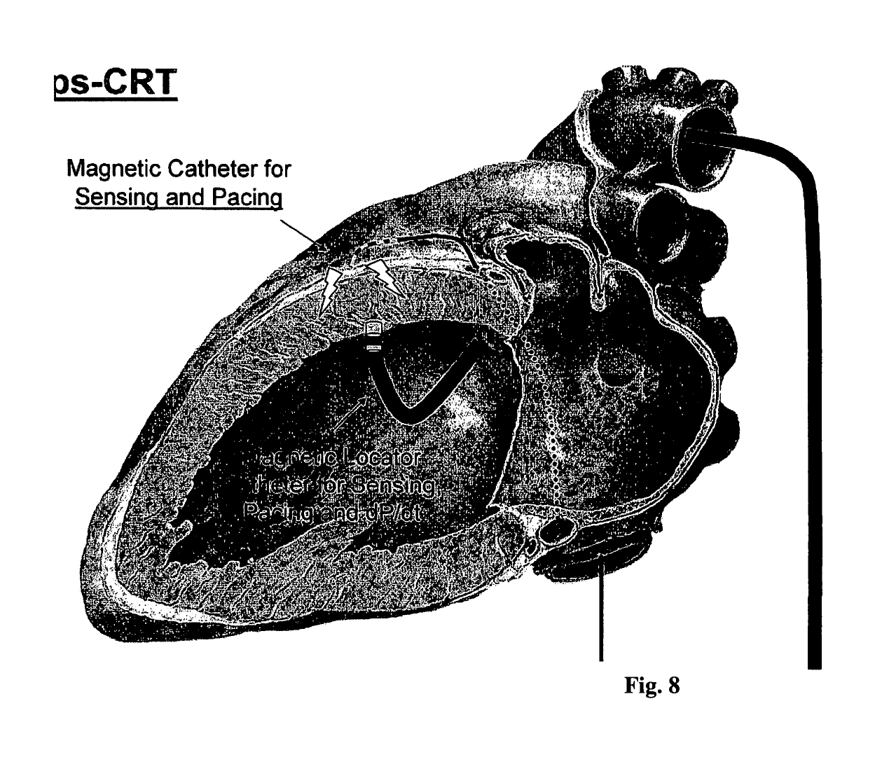

A locator catheter can be placed in the left ventricle using a remote navigation system. In the case of a magnetic navigation system, the locator catheter has a tip that is magnetically responsive. Such a catheter is able to access the posterior and lateral wall effectively. In a preferred embodiment, the locator catheter is also provided with a pressure transducer at the tip, and can pace and sense signals in the left ventricle. FIG. 8 shows an example of a locator catheter in the left ventricle and a magnetic catheter for sensing and pacing that is placed epicardially in the coronary venous vasculature. Thus, for instance, the left ventricle free wall can be analyzed. In the case of a remote magnetic navigation system, the locator catheter can be held in place by a suitably applied external magnetic field. In another preferred embodiment, the locator catheter is anchored in place by means of a screw-tip mechanism that extends out of the distal end of the catheter. The pressure transducer in the locator catheter can measure the rate of change of pressure with respect to time (dP/dt). In particular, the rate of pressure change can be measured as the epicardial left ventricle lead delivers pacing signals.

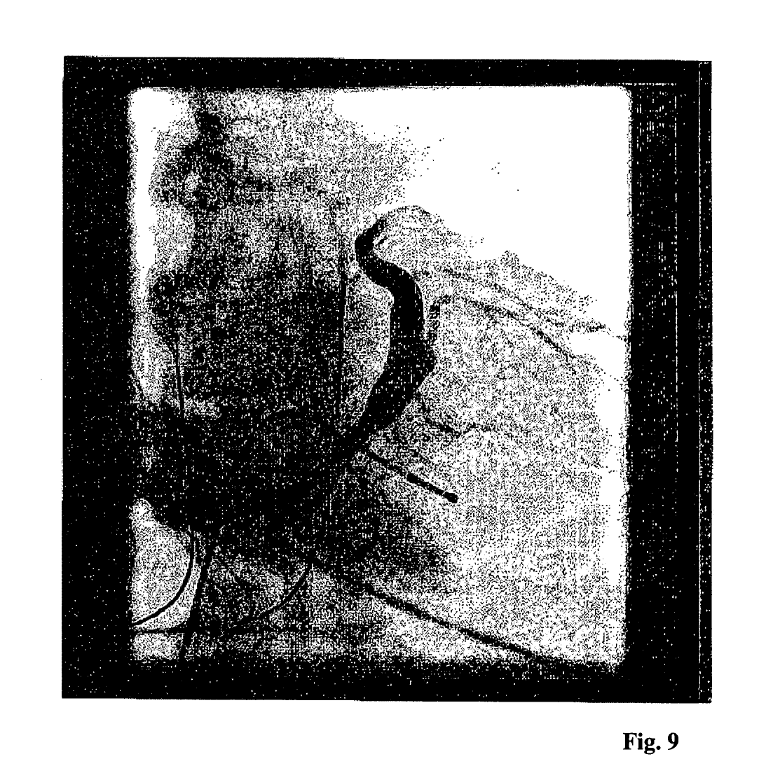

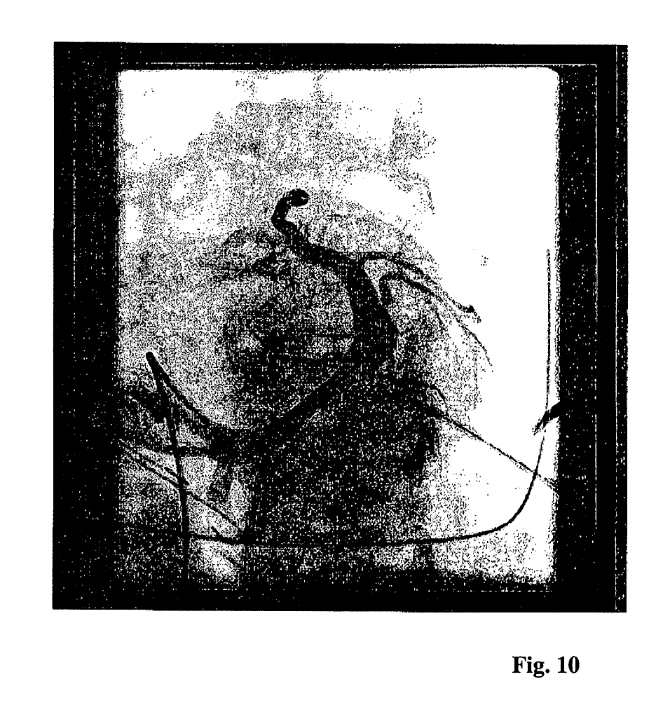

The pacing catheter could be equipped with an electromagnetic location sensor for use with a localization system, whereby the tip position of the catheter within the subject's patient anatomy can be determined. As previously described in U.S. patent application Ser. No. 60/604,101, filed Aug. 24, 2004, for Methods and Apparatus for Steering Medical Devices in Body Lumens (incorporated herein by references) together with at least a pair of X-ray images showing contrast-filled images of the vasculature, such a catheter can be automatically steered and navigated to a destination site by a remote navigation system. A pair of such X-ray images is apparent in FIGS. 9 and 10. From these images, U.S. patent application Ser. No. 60/604,101, filed Aug. 24, 2004, for Methods and Apparatus for Steering Medical Devices in Body Lumens, a three dimensional vascular path or vascular tree can be reconstructed by edge detection image-processing, or by user marking at a set of corresponding points in the at least one pair of X-ray images and the device can be automatically steered by a remote navigation system according to the techniques taught therein. One preferred embodiment of this method employs a magnetic navigation system that applies suitable external fields to orient the device and remotely advance the device either under computer control or by a user-operated input interface such as a joystick. In this case the pacing catheter would incorporate suitable magnetic material in its distal region so that it can respond to an externally applied magnetic field.

In another embodiment of the method, the pacing catheter tip can be localized by image processing methods such as those taught in U.S. patent application Ser. No. 10/977,488, filed Oct. 29, 2004, for Image-Based Medical Device Localization. As the device or catheter is remotely advanced within the vasculature under Fluoro imaging, it is continuously tracked by the image processing algorithms incorporated into the remote navigation system and suitably steered.

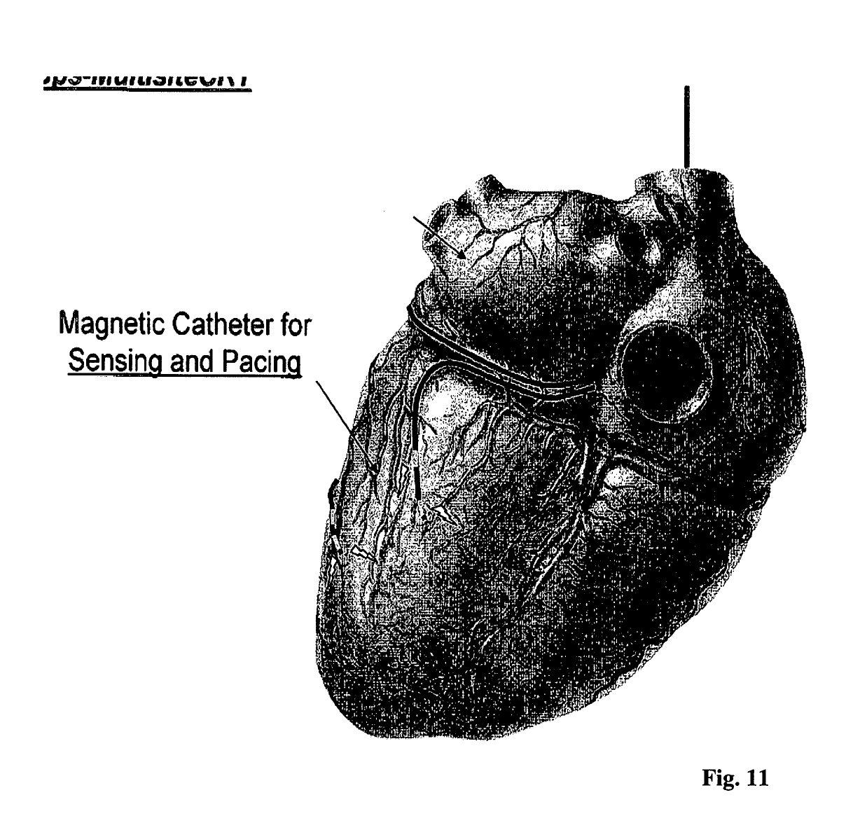

In still another embodiment of the method, multiple pacing catheters could be navigated and placed in multiple locations, as shown in FIG. 11. Each catheter could be left at a given site within the vasculature, where it would remain simply because it is constrained by the vessel walls. Each of these catheters could be navigated automatically, one at a time, by the remote navigation system as described in U.S. patent application Ser. No. 60/604,101, filed Aug. 24, 2004, for Methods and Apparatus for Steering Medical Devices in Body Lumens, and left in place. Subsequently each of these catheters could be used for pacing sequentially or simultaneously in various combinations. The locator catheter would sense the left ventricle signals, and thereafter the pacing catheters can be navigated to alternate sites as desired. An advantage of using multiple pacing catheters is that optimal Sub-Threshold Stimulations can be identified to treat CCM

In still another embodiment, the pacing catheter could be navigated pericardially to a desired site and used to pace the left ventricle.

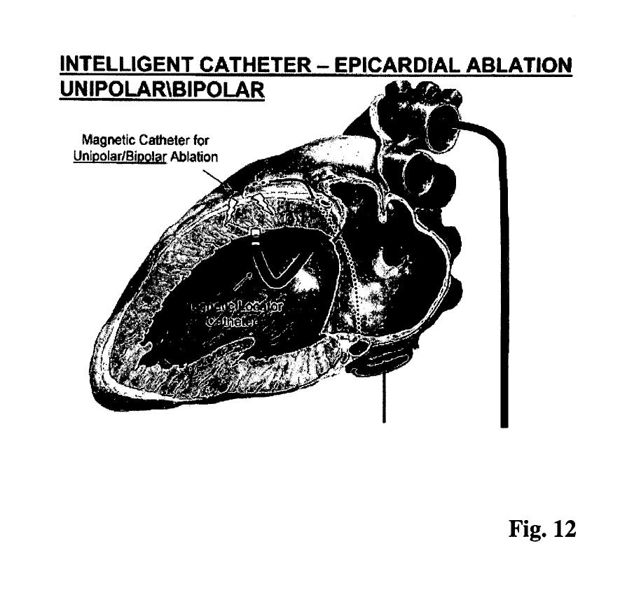

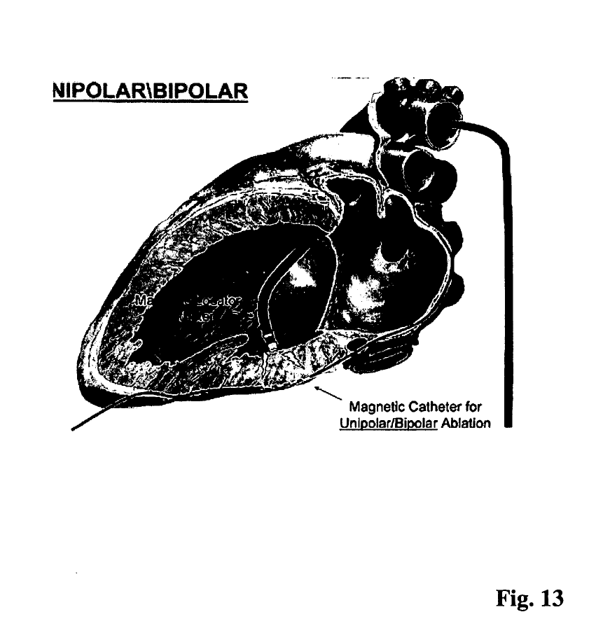

Whether used pericardially or epicardially, in a preferred embodiment the pacing catheter is also an ablation catheter. In this embodiment the location catheter is also endowed with a location sensor for localizing the tip within the patient anatomy. Once it has been placed at a suitable site in the ventricular endocardium, its spatial coordinates are used by the remote navigation system to find the nearest location on a reconstructed three dimensional vascular path. Starting from a known entry point into the coronary venous vasculature, the remote navigation system automatically navigates the pacing catheter through an appropriate vascular path in accordance with the teachings of U.S. patent application Ser. No. 60/604,101, filed Aug. 24, 2004, for Methods and Apparatus for Steering Medical Devices in Body Lumens to place it at this nearest location in the vasculature. Now the electrodes of the pacing catheter and the locator catheter are spatially close together. At this point ablation energy can be delivered to the tissue either in bipolar mode (so that the ablation current flows across the endocardial tissue between the electrodes of the pacing catheter and the locator catheter), or in unipolar mode (with the use of a cutaneous patch, so that the ablation current flows between the locator catheter electrode and a cutaneous patch electrode placed externally on the patient). Bipolar ablation can deliver more energy locally and is expected to result in more effective ablation and shorter ablation times. This is illustrated in FIGS. 12 and 13.

In order to find the best site that couples both electrical and mechanical effects, Pressure-Volume data (PV loops) can be integrated into the remote navigation system. In a preferred embodiment, a 7 “French” (2.33 mm diameter) “over the wire” conductance catheter can be provided with a pigtail and a solid-state pressure transducer to measure several segmental left ventricle volumes (in practice, up to about 7) and pressures from apex to base, as well as total left ventricle volume and net pressure. The left ventricle free wall can be analyzed for the best region to be paced, as follows. Temporary pacing electrodes are placed in the right atrium (RA), right ventricle apex and multiple left ventricle sites. Right atrium pacing is performed at a rate approximately 10% higher than the native sinus rate. Left ventricle hemodynamic data (PV data) is collected during pacing from each electrode and electrode combination employed in the test sequence. All ventricular pacing steps incorporate right atrium stimulation with multiple atrial-ventricular delay intervals set 5-20 ms shorter than the natural AV delay. Each isolated pacing step in the sequence typically lasts for 15 seconds. The data that is collected includes: Ventricular pressures, Ventricular volumes, and rate of pressure change (dP/dt). The conductance volume catheter can be calibrated by using a standard Swan-Ganz thermodilution catheter. The conductance stroke can be matched with the thermodilution SV, followed by removal of the Swan-Ganz catheter after calibration.

After calibration, lead positioning is tested. Aortic pressure, central venous pressure, pulmonary artery pressure and radial artery pressure are all monitored, as also left ventricle stroke volume, conductance catheter and pulse contour. LV Pressure-Volume loops are also monitored, as well as diastolic and systolic volumes, ejection fraction, intra-ventricular mechanical dyssynchrony indices, peak |dP/dt|, peak ejection fraction and peak filling rate. At least 3 different left ventricle settings, followed by 3 dual lead left ventricle settings, followed by best left ventricle setting at 3 different AV delays, best setting combined with 3 different right ventricle lead positions are determined in sequence, for a total of 12 pacing sequences. From this, the best lead positions are determined as follows.

The best lead positions (between one and three, typically) are determined from analyzing the monitored variables for an estimate of mechanical performance of the heart. This can be done manually by a physician recording either mentally or otherwise the Pressure-Volume and associated variables for each setting, or directly entering the recorded variables on a user interface of a remote navigation system. The user can then select the best lead positions from the recorded variables.

Alternatively, the recorded Pressure-Volume and associated real-time variables can be integrated into a remote navigation system. The remote navigation system constructs a cost function from the recorded variables. Recorded variables, whether recorded manually or automatically in a remote navigation system that interfaces with an ECG system and a PV-monitoring system, include: pacing thresholds, sensing amplitude, lead stability, dP/dt, PV loop data, echocardiogram, QRS width of the ECG signal, and others known to those skilled in the art of electrophysiology.

This cost function provides a quantitative measure of the mechanical performance of the heart and includes area W under the Pressure-Volume loop (which is the work performed by the heart during a cardiac cycle). A typical cost function could take the form:
C=a₁*(|dP/dt|ₘₐₓ−b₁)²−a₂*W*W-a₃*Pₘₐₓ*Pₘₐₓ
where the a's are weights which serve to normalize the variables, b₁ is an ideal value for the maximum rate of pressure change, and Pₘₐₓ is the maximum pressure. The remote navigation system compares the cost functions resulting from cardiac cycles at each lead position and thence determines the highest scoring ones.

Thence, bi-ventricular setting responses at the best lead positions using 3 different RV-LV intervals and 3 different AV delays (9 combinations) are also determined. Thus, an optimal combination of both lead positions and setting responses is determined, yielding an optimized set of variables for optimal restoration of both electrical and mechanical function of the heart.

One advantage of using a remote navigation system for determination of best pacing site(s) is that such a system can accurately return to a previously visited position for further data collection or checks. In the context of a magnetic navigation system, as described in U.S. patent application Ser. No. 60/583,855, filed Jun. 29, 2004, Localization of Remotely Navigable Medical Device Using Control variable and Length, incorporated herein by reference, the magnetic field vector and the length of device advancement from a known reference position/length can be repeatedly applied as control variables to yield reproducible return to a desired device tip position. As taught in the above U.S. patent application, the magnetic field vector and catheter length can be stored in the magnetic navigation system when the catheter tip is at a specific location, thereby serving to uniquely identify that spatial location. In this manner, after several sites have been explored, the recorded variables or a cost function associated with the various sites can be stored, and the device can be easily re-navigated to the site that yielded the best results. A fresh comparison of different sites can also be performed easily in this manner. This re-navigation can either be automatically performed by the remote navigation system under computer control, or driven by the user by manual control of the remote navigation system.

It is worth noting that while some of the examples above are in the context of a remote magnetic navigation system, the actuation method actually used by the remote navigation system could take various forms and is not constrained in any manner. For example, other remote navigation methods could employ mechanical pull wires controlled by servo motors, electrostrictive actuation, hydraulic actuation, and such other actuation schemes known to those skilled in the art.

Likewise, the techniques actually used in the methods detailed above could use varying levels of automation, from fully manual control to semi-automated control to fully automated control of the device steering and data recording elements.

1. A method of selecting a site for placing a pacing lead in the heart, the method comprising moving an electrode catheter successively to a plurality of possible placement sites, and at each site determining whether the tissue at the placement site is viable, and if the tissue at the site is viable, applying a pacing signal to the tissue at the site, and measuring the effectiveness of the pacing from the site; and subsequently selecting at least one of the sites which exceeded a predetermined level of pacing effectiveness.

2. The method according to claim 1 wherein at least one of the sites selected corresponds to the greatest pacing effectiveness.

3. The method according to claim 2 wherein the electrode catheter is moved with a remote navigation system from site to site.

4. The method according to claim 1 wherein the remote navigation system is a magnetic navigation system, and wherein the electrode catheter has at least one associated magnetically responsive element.

5. The method according to claim 1 wherein the at least one magnetically responsive element is part of electrode catheter.

6. The method according to claim 1 wherein the at least one magnetically responsive element is part of a guide wire extending through a lumen in the electrode catheter.

7. The method according to claim 1 wherein the electrode catheter has a bi-polar configuration.

8. The method according to claim 1 wherein the electrode catheter has a multi-polar configuration.

9. The method according to claim 1 wherein the electrode catheter has a unipolar configuration.

10. The method according to claim 1 wherein measuring the effectiveness of the pacing comprises measuring the electrocardiogram wave form at a location remote from the site.

11. The method according to claim 1 wherein measuring the effectiveness of the pacing comprises measuring the PV loop.

12. The method according to claim 1 wherein determining tissue viability at the site comprises measuring electrical activity at the site.

13. A method of selecting a site for placing a pacing lead in the heart, the method comprising moving a pacing electrode catheter successively to a plurality of possible placement sites, applying a pacing signal to the tissue at the site, and measuring the effectiveness of the pacing from the site by recording a combination of mechanical and electrical data with a second locator catheter placed in a Ventricle; and subsequently selecting at least one of the pacing sites which exceeded a predetermined level of pacing effectiveness.

14. The method of claim 13 wherein the step of moving the pacing electrode catheter is performed with a remote navigation system.

15. A method for selecting at least two sites for placing at least two pacing leads in the heart for multi-site pacing, the method comprising using a remote navigation system to navigate each of at least two pacing catheters to at least one possible placement site, applying simultaneous pacing signals to the tissue at the respective sites, and measuring the effectiveness of the pacing from the sites by recording a combination of mechanical and electrical data with a second locator catheter placed in a Ventricle.

16. The method of claim 15, where the pacing includes delivery of sub-threshold stimulation signals.

17. A method for selecting at least two sites for placing at least two pacing leads in the heart for multi-site pacing, the method comprising using a remote navigation system to navigate each of at least two pacing catheters to at least one possible placement site, applying sequential pacing signals to the tissue at the respective sites, and measuring the effectiveness of the pacing from the sites by recording a combination of mechanical and electrical data with a second locator catheter placed in a Ventricle.

18. The method of claim 17, where the pacing includes delivery of sub-threshold stimulation signals.

19. A method for selecting a best site for pacing, the method comprising the steps of: navigating a pacing device to different coronary vascular sites, pacing at each site, and recording electrical and mechanical data at at least one ventricular site with each pacing on a user interface.

20.-25. (canceled)
